(19) United States
(12) Patent Application Publication
(10) Pub. No.: US 2010/0056960 A1
(43) Pub. Date: Mar. 4, 2010

(54) Device and method for capturing a gait pattern

(75) Inventor: Michael Lanny, Bad Wildbad (DE)

Correspondence Address: RABIN & Berdo, PC, 1101 14TH STREET, NW, SUITE 500, WASHINGTON, DC, 20005, US

(73) Assignee: MLS LANNY GMBH, Bad wildbad (DE)

(21) Appl. No.: 12/514,362

(22) PCT Filed: Nov. 10, 2007
(86) PCT No.: PCT/EP07/09750

§ 371 Date: May 11, 2009

(30) Foreign Application Priority Data
Nov. 10, 2006 (DE) 10 2006 053 348.8
Nov. 10, 2006 (DE) 20 2006 017 301.3

(51) Int. Cl. A61D 99/00; A61B 5/11

(52) U.S. Cl. 600/592

(57) ABSTRACT

The invention relates to a device for capturing a gait pattern of a living being, comprising a tread plate (4) and at least one sensor (5), associated with the tread plate (4) or integrated into it, a sensor signal of the at least one sensor (5) being supplied to an evaluation device (A). The device (1) further comprises a treadmill (6) which is superposed to the tread plate (4), the at least one sensor (5) allowing the individual tread pulses of a dynamic sequence of movements consisting of a sequence of individual movements to be detected and the sensor signals (S) correlated with the individual tread pulses to be supplied to the evaluation device (A). The invention is characterized in that the tread plate (4) is elastically received in the device (1) by means of a suspension (3), that the one sensor or at least one of the sensors (5) is configured as a sensor (5) detecting an acceleration and/or a deviation of the tread plate (4).

The present invention relates to a device for capturing a gait pattern of a living being, especially of a hoofed animal, and here especially of a horse, comprising a tread plate and at least one sensor, associated with the tread plate or integrated into it, a sensor signal of the at least one sensor being supplied to an evaluation device, wherein the device comprises a treadmill which is superposed to the tread plate, and the at least one sensor allows the individual tread pulses of a dynamic sequence of movements, consisting of a sequence of individual movements, to be detected and the sensor signals correlated with the individual tread pulses to be supplied to the evaluation device, and to a method for capturing the gait pattern of a living being, especially of a hoofed animal, and here especially of a horse, wherein any application of pressure to a tread plate is captured by at least one sensor, and the sensor signal resulting from the application of pressure to the tread plate is supplied to an evaluation device, wherein the living being, for capturing individual tread pulses out of a dynamic sequence of movements that consists of a sequence of tread pulses, is placed on a treadmill which is superposed to the tread plate, and wherein the individual tread pulses of a dynamic sequence of movements, consisting of a sequence of individual movements, is captured and the sensor signals correlated with the individual tread pulses are supplied to the evaluation device.

Devices and methods of that kind have been known, for example, from human medicine and orthopedics and are used for detecting the stress situation of a patient's feet. The patient is placed for this purpose on a tread plate, designed as load-measuring device, and the load so applied on the tread plate is detected by a plurality of sensors configured as flat elements to determine the static loading of the tread plate produced by the patient's feet. In this way, the device captures an area-related load distribution pattern of the patient's foot placed on the tread plate, and the sensor signals are supplied to the evaluation device where the load pattern of the foot so obtained is displayed and/or evaluated to derive information on the load centers of the particular foot standing on the tread plate.

The known devices and methods are, however, connected with the disadvantage that they only permit a static gait analysis with the user standing on the tread plate of the device. It is a disadvantage that it is not possible with the known devices and methods to analyze a dynamic motion sequence, i.e. a sequence composed of a plurality of tread sequences.

The need to analyze such a dynamic motion sequence is encountered not only in the field of human medicine, especially in orthopedics or sports medicine. Specifically, it is desirable also in connection with animals, especially with horses, to have a method and a device that allow a dynamic motion sequence to be analyzed via a captured tread pattern. Till now, especially lameness examinations of horses could be performed only visually: For performing a lameness examination the veterinarian till now had to observe the movements of the horse moving at walking pace and to then rely on his visual impression so gained.

A device and a method of the before-mentioned kind have been known from WO 93/06779. That printed publication describes a device comprising a tread plate with a plurality of associated sensors where the sensor signals can be supplied to an evaluation device. A treadmill is arranged on top of the tread plate. The sensors used are force sensors arranged below the treadmill. Such a device presents the disadvantage that the tread plate is arranged rigidly in the device. This is a substantial disadvantage especially for lameness examinations of hoofed animals, such as horses, as the rigid design and arrangement of the tread plate creates an “unnatural” ground which may influence the gait of the animal and may thus falsify the examination results.

U.S. Pat. No. 5,186,062 discloses that an extensive measuring surface, having a length of 4 m and a width of 0.8 m, for example, and being arranged below a covering tarpaulin, is formed in a path of movement of a horse. The measuring surface comprises a plurality of measuring sections arranged one parallel to the other, each being shorter than the length of a horse's hoof and wider than the width of a horse. Each measuring section is provided with a force measuring device having a substantially rectangular force measuring surface. The force measuring devices are arranged in a sub-frame and each force measuring device comprises a rigid traverse which, related to the sub-frame, is carried on block gauges on both sides. The load acting on the traverses is measured and the corresponding signals are evaluated. Such a device is connected with the disadvantage that the measuring surface must be very long in this case as the horse has to be guided over the measuring surface for capturing a dynamic motion pattern. As the entire measuring surface has to be covered by the measuring sections, a plurality of force measuring devices is required and their signals must be evaluated. That disadvantage shows particularly clearly if one regards the exemplary embodiment disclosed in the before-mentioned printed publication: With a recommended length of the measuring field of 4 m and a length of the individual measuring sections of 25 mm, 160 measuring sections and, accordingly, 160 force measuring devices would be required, and the evaluation device must be capable in this case of evaluating 160 measuring signals in parallel. Such an apparatus input is not tolerable under economical aspects.

It is the object of the present invention to improve a device and a method of the before-mentioned kind so that improved capturing of the gait pattern of a dynamic motion sequence is rendered possible by simple means.

The device according to the invention proposes to solve that object by an arrangement where the tread plate is supported elastically in the device and where the sensor, or at least one of the sensors used is an acceleration sensor or a displacement sensor for capturing the acceleration and/or excursion of the tread plate produced by forces applied on the tread plate by the user's extremities.

These features according to the invention advantageously provide a method and a device that distinguish themselves by improved capturing of tread pulses of a dynamic motion sequence. The elastic suspension of the tread plate and, accordingly, of the treadmill arranged on the latter, causes the tread plate to yield under the action of the user's extremity. This advantageously permits to simulate natural ground conditions which causes especially horses or similar animals to move in their habitual way. The use of an acceleration sensor or a displacement sensor then allows the acceleration of the tread plate produced by the user's extremities and/or the excursion of the tread plate to be captured. Instead of capturing the force acting on the tread plate, the device according to the invention therefore advantageously captures the acceleration and/or excursion of the tread plate as such, as they are produced by the forces applied on the plate by the movements of the user's extremities. To proceed in this way provides the advantage of improved accuracy and a simpler structure, compared with the use of a plurality of force sensors which, in addition, would be continuously loaded by the user standing on the tread plate. Further, it is no longer necessary in the case of the device according to the invention to provide a plurality of sensors as in principle a single sensor will be sufficient to capture the acceleration and/or the excursion of the tread plate. The features according to the invention therefore not only allow a static load situation to be analyzed, but also and advantageously provide the possibility, by simply capturing the corresponding tread pulses, to analyze a simultaneous sequence of motion phases, i.e. a dynamic sequence of movements.

Another advantageous further development of the invention provides that the stiffness of the tread plate suspension can be flexibly adjusted. This provides the possibility, for example, to adapt the excursion of the tread plate to the specific demands of a user, for example by selecting a higher degree of stiffness for users of higher weight, or a lower degree of stiffness for users of lower weight. Also, it is possible in this way to simulate different walking grounds, a higher degree of stiffness simulating a harder ground and a lower degree of stiffness simulating a softer ground.

An advantageous further development of the invention provides that the sensor signals correlated with the individual tread pulses of a dynamic sequence of movements can be displayed on a display unit of the evaluation device, the evaluation device preferably generating a signal when a tread pulse deviates from another tread pulse and/or from a predefined standard tread pulse. In this way, diagnoses of false stress conditions can be established in an especially simple way, even when the device is operated by an inexperienced operator.

An advantageous further development of the invention provides that the device comprises a correlation device by which the sensor signal of the sensor, or of at least one of the sensors, can be clearly assigned to a specific extremity of the user. With the aid of such a correlation device it is possible—for example via a pulse triggered by a light barrier and a reflector attached to one extremity of the user—to identify a sensor signal as directly coming from that extremity. Knowing the step sequence performed by the extremities it is then possible to correlate all tread patterns with the respective extremities of a user.

Another advantageous further development of the invention provides that the tread plate suspension comprises at least one bearing with a first bearing part and a second bearing part connected in parallel. Preferably, the second bearing part exhibits a low degree of stiffness while the first bearing part is given a variable stiffness. As a result of that variable stiffness of the first bearing part the overall stiffness of the bearing is variable as well.

Another advantageous further development of the invention provides that the first bearing part comprises a first bearing element of high stiffness connected in series with a second bearing element of variable stiffness. Preferably, the second bearing element of the first bearing part is configured as an air pad. It is then possible, by varying the air pressure in the air pad, to vary the longitudinal extension of the pad and, accordingly, the contact pressure of the second bearing element of the first bearing part relative to the tread plate. One thereby achieves the desired variability of the stiffness of the first bearing part and, accordingly, of the entire suspension.

According to another advantageous further development of the invention the tread plate is a light structure. This is an advantage especially for light-weight users, whose own weight is negligible, related to a heavy tread plate, so that only little excursion and acceleration of the tread plate would occur. Designing the tread plate as a light structure increases the influence of the user's weight and, accordingly, allows more meaningful tread analyses to be obtained.

Another advantageous further development of the invention provides that the tread plate can be tilted in the device. Preferably, the device is provided for this purpose with at least one frame support element in which a frame, supporting the tread plate, is seated for rotation about an axis, transversely to a walking direction, and at least a second frame element, which can be adjusted in length for varying the spacing between a ground bearing point and a frame bearing point of the second frame support element and, accordingly, the inclination of the tread plate. Preferably, the first frame support element is arranged on the frame in a position offset from an axis of symmetry, on the side of the axis of symmetry opposite the second frame support element. Compared with a central arrangement, that way of supporting and especially that eccentric arrangement of the first frame support element prevent tilting of the device about its axis and guarantee the necessary stability of the device.

Other advantageous further developments of the invention are the subject-matter of the sub-claims.

Further features and advantages of the invention will be apparent from the embodiment described in the following, with reference to the drawings. Specifically, the drawings show:

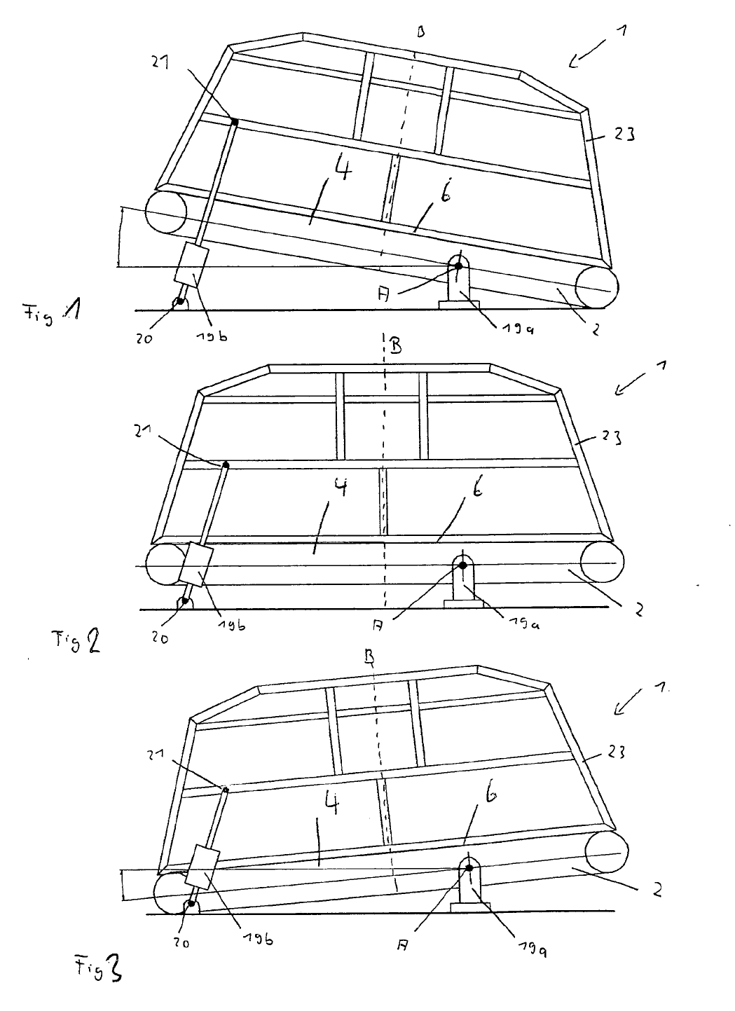

FIG. 1 a side view of one embodiment of a device for capturing a tread pattern, in an upwardly tilted position;

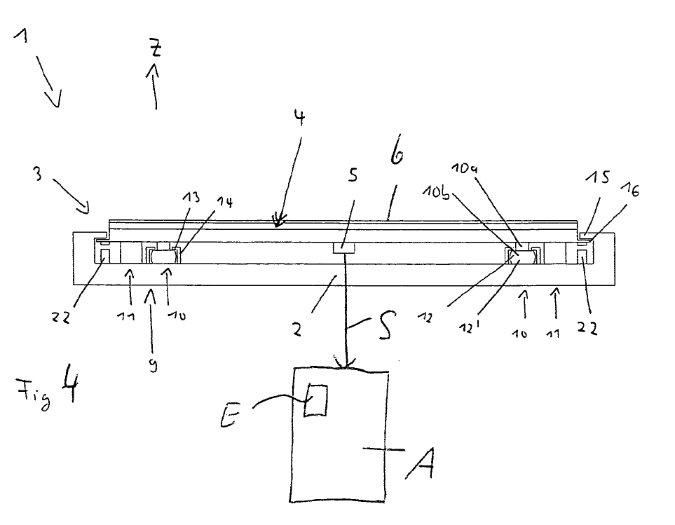

FIG. 2 a side view of the embodiment illustrated in FIG. 1, in horizontal position;

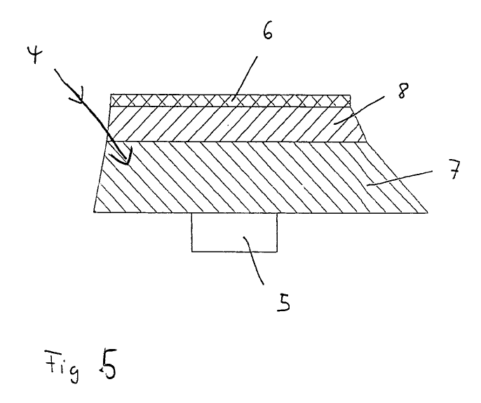

FIG. 3 a side view of the embodiment illustrated in FIG. 1, in a downwardly tilted position;

FIG. 4 a cross-section through the embodiment illustrated in FIG. 1;

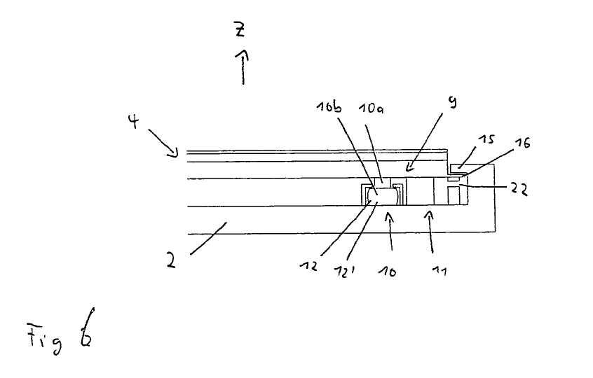

FIG. 5 an enlarged cross-sectional view of a tread plate of the embodiment illustrated in FIG. 1;

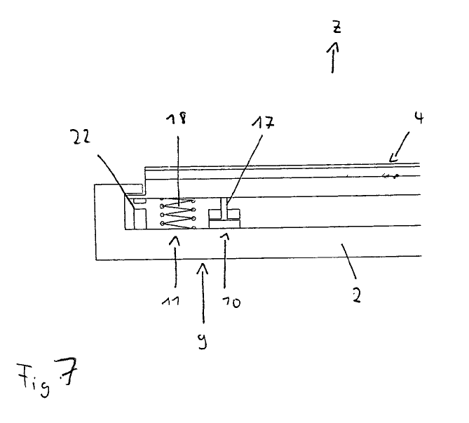

FIG. 6 an enlarged cross-sectional view of a suspension of the embodiment illustrated in FIG. 1; and

FIG. 7 an alternative embodiment of a suspension of the embodiment.

The Figures now show one embodiment of a device 1 for capturing a gait pattern of a living being, especially of a horse. It shows a tread plate 4, supported in a frame 2 via an elastic suspension 3 that will be described hereafter in more detail, with one or more sensors 5 (see FIG. 4)—configured as acceleration sensors in this case—arranged below that suspension.

Now, in order to not restrict the device—as is the case with the known devices to performing static analyses of the user's movements, but to instead provide the possibility to analyze the individual tread patterns of a dynamic motion sequence, the arrangement of the described device 1 is such that it comprises a treadmill 6 arranged on top of the tread plate 4, on which the user performs that dynamic sequence of individual motion processes—when the user is a horse, for example, a sequence of walking, trotting or galloping movements—that results in corresponding tread pulses, which individual tread pulses of the user then result in corresponding sensor signals of one or more sensors 5: Now, when an extremity of a user hits upon a tread plate 4, the latter is deflected, due to its elastic suspension 3, and its acceleration is detected by the one or more sensors 5. The sensor signals S of the one or more sensors 5 are evaluated by an evaluation device A, and are preferably displayed on a display unit E of an evaluation device A.

The at least one sensor, and preferably all sensors 5 are configured as a single-axial or multi-axial acceleration sensor so that the acceleration of the tread plate 4, provoked by the forces applied on it by the user's extremities, can be detected. However, that operating principle of the sensor 5 is not a compulsory requirement. There is also the possibility to provide a displacement sensor which guarantees that the excursion of the tread plate 4, provoked by the forces applied on it by the user's extremities, can be detected.

In cases where the device 1 comprises a single sensor 5, the latter preferably will be arranged centrally below the tread plate 4. Where more than one sensors 5 are provided, the tread movements acting on the tread plate 4 can then be recorded as an area-related motion pattern. Preferably, the device 1 comprises in this case either two or four or six sensors 5, which two or four or six sensors are assigned to the front or rear half of the tread plate 4, respectively, or to one quadrant or to the ends and the center of the tread plate 4, respectively. This allows by simple means to separately record the tread pulses produced by the forefeet or the hind extremities of the user. If four or more sensors 5 are provided, each tread pulse produced by an extremity of a four-legged user can be detected separately.

As, depending on the gait, especially that of horses, the sequence of motion phases will consist either of a four-beat movement (walk) or a two-beat movement (trot, gallop), it is possible without any difficulty to correlate the individual tread pulses detected with a specific extremity of the user, provided the correlation of one given tread pulse with one given extremity is known. That correlation may be achieved by visual observation. However, there is also the possibility to equip the device 1 with a correlation device not shown—for example a light barrier, which is triggered by the movement of a specified extremity of the user—for example the left forefoot. The output signal of that correlation device is then supplied to the evaluation device A, and the time-correlated signal of the sensor 5 is then assigned to that extremity of the horse that produced the output signal of the triggering unit. The motion sequence of the extremities being known, it is then easily possible to correlate the next tread pulses with the different extremities.

These features on the one hand allow a sequence of tread patterns to be detected during a sequence of movements to be observed, and on the other hand simultaneously permit that sequence of movements to be observed by an observer—while the tread pattern is being recorded by the device 1—so that the information gained from that tread pattern analysis can be easily supplemented by the findings derived by an observer—for example a veterinarian, therapist or trainer—from his optical impression.

The sequence of tread patterns so gained now advantageously gives a treating physician, or a therapist or trainer, not only the possibility to analyze a static load situation produced by the user's extremities acting on the tread plate 4—as is the case with known devices—but provides an advantageous way of detecting and evaluating a sequence of motion phases—for example when the user treads on the tread plate 4 or during other movements performed by the user—and to then use the information so gained to determine the course of therapeutic, diagnostic, recovery or training programs.

The described device 1 thus advantageously allows lameness examinations of horses to be carried out easily by placing the horse on the treadmill 6 and then causing the horse to perform the desired movement—walk, trot or gallop. The tread pulses produced by those movement processes and acting on the tread plate 4 are then detected by the sensor 5 whose sensor signals S are supplied to the evaluation device A where they are analyzed and displayed on the display unit E of the evaluation device A. By comparing the individual tread pulses one with the other and/or with predefined tread pulses that represent the normal condition, for example the tread pulse of a healthy horse's foot, any deviations indicative of a false load pattern produced by the horse, for example due to lameness and/or other irregularities, can be easily detected.

Preferably, the evaluation device A produces a signal when a deviation is encountered between a detected tread pulse and another tread pulse and/or a predefined standard tread pulse. Such a feature provides the advantage that false strain of the horse can be detected in this case even by an inexperienced operator of the device.

In order to ensure that the treadmill 6 will run on the tread plate 4 as smoothly as possible, it is preferably provided that the treadmill 6—as shown in FIG. 5—moves on a lining 8 provided on a base plate 7 of the tread plate 4.

Preferably, the treadmill 6 runs continuously. The belt return system necessary in this case is not shown in the Figures, but is well known to the man of the art.

The tread plate 4 is received in an elastic suspension 3. This provides the advantage that the resistance opposed by the tread plate 4 to the user's extremities can be easily varied and adjusted to the particular user.

The suspension 3, which is illustrated in detail in FIGS. 6 and 7, is composed of a plurality, preferably four, bearings 9 each of which comprises a first bearing part 10 and a second bearing part 11 that are connected in parallel one to the other and that are connected with both the frame 2 and the tread plate 4. The first bearing part 10 is configured as a series connection of a first bearing element 10a and a second bearing element 10b.

Both the second bearing part 11 and the first bearing element 10a of the first bearing part 10 are configured as rubber elements of uniform thickness, the rubber element of the second bearing part 11 having a lower stiffness and the rubber element of the first bearing element 10a of the first bearing part 10 having a higher stiffness. On the other hand, the second bearing element 10b of the first bearing part 10 consists of an air pad 12 in the form of air bellows 12′ whose air volume and, accordingly, air pressure can be adjusted by a control unit not shown. The second bearing part 11 of the bearing 9 therefore has a uniform stiffness, while the first bearing part 10 has a variable stiffness. That variability is achieved by the variable air pressure in the air pad 12 as an increase of the air pressure causes the air pad to expand in length, in a direction Z, and to thereby increase the contact pressure of the first bearing element 10a of the first bearing part 10 acting on the tread plate 4, whereas at lower air pressure the second bearing part 11 of low stiffness is active alone to support the main portion of the tread plate 4. Accordingly, the overall stiffness of the bearing 9 results from the interaction between the first bearing part 10 of variable stiffness and the second bearing part 11 of constant stiffness.

With a view to preventing unrestricted uncontrolled movements of the tread plate 4 in Z direction, and any asymmetrical lifting, the extension of the air pad 12 in lengthwise direction is limited toward the top by a stop surface 13 of a mount 14. However, by further increasing the air pressure in the air pad 12, it is possible to further increase its stiffness, through the accompanying increase of its modulus of elasticity E.

Due to the variable stiffness of the bearings 9 of the suspension 3 it is now possible to adapt the device 1 to the particular weight of the respective user by increasing the stiffness for a user of higher weight, or reducing the stiffness for a user of lower weight. In this way, the excursion of the tread plate 4 can be adjusted, and the measuring range of the one sensor or the more sensors 5 can be utilized over a wide range. Preferably, the device 1 is provided for this purpose with—in the present embodiment—four displacement sensors 22 provided at the corners of the tread plate 4, through which the excursion of the tread plate 4 can be detected for then adjusting the stiffness of the suspension 3.

Further, the flexibly adjustable stiffness of the suspension 3 also allows different walking grounds to be simulated, a high stiffness of the suspension 3 simulating a hard walking ground and a low stiffness of the suspension 3 simulating a soft ground.

In order to avoid uncontrolled movements of the tread plate 4 in Z direction, for example when the user steps down from the tread plate 4 or lifts one extremity, the frame 2 is provided with a projection 15 that will act as a stop for an end stop element 16 of the tread plate 4 when an excessive movement occurs in the Z direction, thereby limiting that movement.

The described suspension 3 provides the possibility to give each of the bearings 9 of the suspension 3 a different stiffness, for example for focused training of the forelegs or hind legs or the right or left extremities of the horse, or, during a recovery phase of a horse, for building up an injured extremity in a focused way.

In the illustrated embodiment, the Z direction corresponds substantially to the line of gravity although some inclination of the device 1, for example to simulate a movement up or down a hill, is of course likewise imaginable.

To this end, the device 1 is provided with a frame support 9—as illustrated in the side views of FIGS. 1 to 3. The frame support consists of a first frame support element 19a, in which the frame 3 is seated to rotate about an axis A transversely to the walking direction, and at least one second frame support element 19b of adjustable length. The second frame support element 19b is connected with the ground via a ground bearing point 20 and with a railing 23 via a frame bearing point 21 so that the variable length of the second frame bearing element 19b makes the spacing of the ground bearing point 20 and the frame bearing point 21 variable and, thus, the inclination of the device 1 adjustable. Preferably, the first frame bearing element 19a is arranged in a position offset from the axis of symmetry B, on the side of the axis of symmetry B opposite the second frame bearing element 19b. Compared with a central arrangement, this improves the stability of the tread plate 4 as tilting about the axis A is prevented or hindered in this way. Preferably, the device 1 comprises two frame bearing elements 19a, each enclosing the axis A, and two frame bearing elements 19b arranged one behind the other in the illustration.

When the device 1 is primarily used by light-weight users, their own weight does not count much compared with a relatively heavy tread plate 4 so that only slight excursions of the tread plate 4 will be encountered and no satisfactory tread pattern can be determined. It is preferred for this reason to design the tread plate 4 in this case as a light structure, known to the man of the art, for example as a honeycomb structure whereby the influence of the user's weight, relative to the weight of the tread plate 4, will be increased and more pronounced tread pulses will be obtained.

As an alternative to the described embodiment of the bearings 9 of the suspension 3, other embodiments are of course also possible, for example the one illustrated in FIG. 7 where the first bearing part 10 is designed as an adjustable hydraulic damper 17 and the second bearing part 11 is designed as a spring 18 connected in parallel with the latter. The variable stiffness of the bearing 9 is guaranteed in this case by the adjustable damping effect of the hydraulic damper 17.

1. Device for capturing a gait pattern of a living being, comprising a tread plate (4) and at least one sensor (5), associated with the tread plate (4) or integrated into it, a sensor signal of the at least one sensor (5) being supplied to an evaluation device (A), wherein the device (1) comprises a treadmill (6) which is superposed to the tread plate (4), and wherein the at least one sensor (5) allows the individual tread pulses of a dynamic sequence of movements, consisting of a sequence of individual movements, to be detected and the sensor signals (S) correlated with the individual tread pulses to be supplied to the evaluation device (A), characterized in that the tread plate (4) is elastically supported in the device (1) by a suspension (3) and that the sensor, or at least one of the sensors (5) is a sensor that captures an acceleration and/or excursion of the tread plate (4).

2. The device as defined in claim 1, characterized in that the device comprises a sensor (5) that covers the whole tread plate (4) or, two sensors (5) each of which is assigned to one half of the tread plate (4) or, four sensors (5), each assigned to one quadrant of the tread plate (4).

3. (canceled)

4. (canceled)

5. The device as defined in claim 1, characterized in that the sensor signals (S) correlated with the individual tread pulses of the dynamic sequence of movements can be displayed on a display unit (E) of the evaluation device (A) and that the individual tread pulses of the dynamic sequence of movements can be compared one with the other and/or with predefined standard tread pulses by the evaluation device (A).

6. The device as defined in claim 5 claim 1, characterized in that a signal can be produced by the evaluation device when a tread pulse deviates from another tread pulse and/or from a predefined standard tread pulse.

7. The device as defined in claim 1, characterized in that the device (1) comprises a correlation device by which a sensor signal (S) of the sensor or the at least one of the sensors (5) can be clearly correlated with a given extremity of a user of the device (1).

8. The device as defined in claim 1, characterized in that the stiffness of the suspension (3) of the tread plate (4) can be adjusted.

9. The device as defined in claim 1, characterized in that the suspension (3) of the tread plate (4) comprises at least one bearing (9) in which a first bearing part (10) and a second bearing part (11) are connected in parallel.

10. The device as defined in claim 9, characterized in that the second bearing part (11) has a low stiffness while the first bearing part (10) has a variable stiffness.

11. The device as defined in claim 9, characterized in that the first bearing part (10) comprises a first bearing element (10a) of high stiffness which is connected in series with a second bearing element (10b) of variable stiffness.

12. The device as defined in any of claim 9, characterized in that the second bearing element (10b) of the first bearing part (10) is configured as a pneumatic, hydraulic, mechanical or electromagnetic spring means.

13. The device as defined in claim 12, characterized in that the spring means is configured as an air pad (12).

14. The device as defined in claim 13, characterized in that the spring means (12) is provided with a feature limiting the extension in length in the vertical direction (Z).

15. The device as defined in claim 14, characterized in that the feature limiting the extension in length of the air pad (12) as configured as a stop surface (13) of a mount (14).

16. The device as defined in claim 9, characterized in that the first bearing part (10) is an adjustable hydraulic damper (17) and the second bearing part (11) is a spring (18).

17. The device as defined in any of the preceding claims, claim 1, characterized in that the tread plate (4) comprises a feature limiting the mobility in a vertical direction (Z), which is in particular configured as a projection (15) of a frame (2) of the device (1) and an end stop (16) of the tread plate (4) that can be moved against that projection.

18. (canceled)

19. The device as defined in claim 1, characterized in that the tread plate (4) is designed as a light structure.

20. The device as defined in claim 1, characterized in that the frame (2) is supported in the device (1) for being tilted.

21. The device as defined in claim 20, characterized in that the device (1) comprises a frame support (19) with at least one frame support element—(19) in which a frame (2) is seated for rotation about an axis (A), transversely to a walking direction, and at least one second frame support element (19b), which can be adjusted in length for varying the spacing between a ground bearing point (20) and a frame bearing point (21) of the second frame support element (19b) and, accordingly, the inclination of the tread plate (4).

22. The device as defined in claim 21 characterized in that the first frame support element (19a) is arranged on the frame (2) in a position offset from an axis of symmetry (B), on the side of the axis of symmetry (B) opposite the second frame support element (19b).

23. Method for capturing the gait pattern of a living being, especially of a horse, wherein any application of pressure to a tread plate (49) of a device is captured by at least one sensor (5), and the sensor signal (S) resulting from the application of pressure to the tread plate (4) is supplied to an evaluation device (A), wherein the living being, for capturing individual tread pulses out of a dynamic sequence of movements that consists of a sequence of tread pulses, is placed on a treadmill (6) which is superposed to the tread plate (4), and wherein the individual tread pulses of a dynamic sequence of movements, consisting of a sequence of individual movements, are captured by the at least one sensor (5) and the sensor signals (S) correlated with the individual tread pulses are supplied to the evaluation device (A), characterized in that the tread plate (4) is elastically supported in the device (1) and that the sensor (5), or at least one of the sensors (5), is an acceleration sensor or a displacement sensor that captures an acceleration and/or excursion of the tread plate (4) produced by the extremities of the user acting on the tread plate (4).
